(19) United States
(12) Patent Application Publication
(10) Pub. No.: US 2024/0050706 A1
(43) Pub. Date: Feb. 15, 2024

(54) Rapidly Insertable Central Catheters Including Catheter Assemblies and Methods Thereof

(71) Applicant: Bard Access Systems, Inc., Salt Lake City, UT (US)

(72) Inventors: Glade H. Howell, Draper, UT (US); Jason R. Stats, Layton, UT (US)

(21) Appl. No.: 18/383,814

(22) Filed: Oct. 25, 2023

Related U.S. Application Data
(63) Continuation of application No. 17/326,017, filed on May 20, 2021, now Pat. No. 11,819,638.
(60) Provisional application No. 63/028,445, filed on May 21, 2020.

(51) Int. Cl. A61M 25/06; A61M 25/00; A61M 25/09

(52) U.S. Cl. CPC A61M 25/0631; A61M 25/007; A61M 25/0097; A61M 25/0693; A61M 25/09; A61M 25/0026; A61M 25/09041; A61M 25/0606; A61M 2025/024

(57) ABSTRACT

Rapidly insertable central catheters (“RICCs”) including catheter assemblies and methods thereof are disclosed. For example, a RICC assembly includes a RICC and an introducer. The RICC includes a catheter tube, a catheter hub, and one or more extension legs coupled in the foregoing order. The catheter tube includes a side aperture that opens into an introducing lumen that extends from at least the side aperture to a distal end of the RICC. The introducer includes a retractable-needle device, a syringe, and a coupling hub that couples the retractable-needle device and the syringe together proximal of the side aperture in a ready-to-deploy state of the RICC assembly. The retractable-needle device includes an introducer needle. A needle tip in a distal-end portion of a shaft of the introducer needle extends beyond the distal end of the RICC when the RICC assembly is in the ready-to-deploy state of the RICC assembly.

### PRIORITY

This application is a continuation of U.S. patent application Ser. No. 17/326,017, filed May 20, 2021, which claims the benefit of priority to U.S. Provisional Application No. 63/028,445, filed May 21, 2020, which is incorporated by reference in its entirety into this application.

### BACKGROUND

A central venous catheter (“CVC”) is formed of a material having a relatively low durometer, which contributes to the CVC having a lack of column strength. Due to the lack of column strength, CVCs are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the number of steps of the Seldinger technique. As such, there is a need to reduce the number of steps and medical devices involved in introducing a catheter such as a CVC into a patient and advancing the catheter through a vasculature thereof.

Disclosed herein are rapidly insertable central catheters (“RICCs”) including catheter assemblies and methods thereof that address the foregoing.

### SUMMARY

Disclosed herein is a RICC assembly. The RICC assembly includes, in some embodiments, a RICC and an introducer. The RICC includes a catheter tube, a catheter hub coupled to a proximal-end portion of the catheter tube, and one or more extension legs, wherein each extension leg of the one-or-more extension legs is coupled to the catheter hub by a distal-end portion thereof. The catheter tube includes a first section, a second section, and a side aperture through a side of the catheter tube in a distal-end portion thereof but proximal of the first section of the catheter tube. The first section of the catheter tube is formed of a first material having a first durometer. The second section of the catheter tube is formed of a second material having a second durometer less than the first durometer. The side aperture opens into an introducing lumen of the catheter tube that extends from at least the side aperture to a distal end of the RICC. The introducer includes a retractable-needle device, a syringe, and a coupling hub coupling the retractable-needle device and the syringe together proximal of the side aperture in at least a ready-to-deploy state of the RICC assembly. The retractable-needle device includes an introducer needle. The introducer needle has a shaft and a needle tip in a distal-end portion of the shaft. The needle tip extends beyond the distal end of the RICC when the RICC assembly is in at least the ready-to-deploy state of the RICC assembly.

In some embodiments, the shaft of the introducer needle extends through a longitudinal through hole of the coupling hub, through the side aperture of the catheter tube, and along the introducing lumen of the catheter tube in at least the ready-to-deploy state of the RICC assembly.

In some embodiments, the coupling hub includes a sealed blood-flashback chamber. A cutout of the shaft of the introducer needle is disposed in the blood-flashback chamber. The cutout is configured to release blood into the blood-flashback chamber upon the needle tip accessing a blood-vessel lumen of a patient.

In some embodiments, the coupling hub includes a side arm extending from a side of the coupling hub. The syringe is fluidly coupled to the blood-flashback chamber by a side-arm lumen of the side arm for aspirating blood upon accessing the blood-vessel lumen.

In some embodiments, the introducer further includes a clip having a syringe-clipping portion and needle device-clipping portion. The syringe-clipping portion of the clip is configured to clip the syringe by a barrel of the syringe. The needle device-clipping portion of the clip is configured to clip the retractable-needle device by a body of the retractable-needle device while allowing the retractable-needle device to slide in the needle device-clipping portion of the clip.

In some embodiments, the syringe includes a plunger disposed in the barrel of the syringe. The plunger includes a plunger extension configured to allow a clinician to withdraw the plunger from the barrel by proximally pushing a tab of the plunger extension while handling the RICC assembly around the coupling hub.

In some embodiments, the coupling hub includes a tab configured to allow a clinician to single handedly advance the RICC over the needle tip with a single finger of a hand while holding a distal-end portion of the retractable-needle device between a thumb and another finger or fingers of the hand.

In some embodiments, the retractable-needle device further includes an access guidewire and an access-guidewire actuator. The access guidewire is disposed in a needle lumen of the introducer needle. The access-guidewire actuator is configured to advance a distal-end portion of the access guidewire beyond the needle tip. The access-guidewire actuator is also configured to withdraw the distal-end portion of the access guidewire into the distal-end portion of the shaft of the introducer needle proximal of the needle tip.

In some embodiments, the access-guidewire actuator includes a slider slidably disposed in a longitudinal slot of a housing of the retractable-needle device. The slider includes an extension coupled to a proximal-end portion of the access guidewire within a cavity of the retractable-needle device enclosed by the housing.

In some embodiments, a proximal-end portion of the introducer needle is disposed in a carriage of the retractable-needle device. When actuated, an introducer-needle actuator of the retractable-needle device is configured to release a compressed compression spring around the carriage and thrust both the carriage and the introducer needle proximally into the cavity of the retractable-needle device.

In some embodiments, the carriage is configured to engage the slider of the access-guidewire actuator when the carriage is thrust proximally into the cavity of the retractable-needle device. The access guidewire is withdrawn into the cavity of the retractable-needle device by the distal-end portion thereof together with the introducer needle when the introducer-needle actuator is actuated.

In some embodiments, the RICC further includes a sterile barrier over the catheter tube between the catheter hub and the side aperture of the catheter tube. The sterile barrier is configured to split apart when a sterile-barrier tab of a proximal-end portion of the sterile barrier is pulled away from the catheter tube by the sterile-barrier tab.

In some embodiments, the RICC includes a set of three lumens including a distal lumen, a medial lumen, and a proximal lumen formed of fluidly connected portions of three catheter-tube lumens, three hub lumens, and three extension-leg lumens. The introducing lumen of the catheter tube is coincident with a distal-end portion of the distal lumen.

In some embodiments, the distal lumen has a distal-lumen aperture in the distal end of the RICC. The medial lumen has a medial-lumen aperture in the side of the catheter tube proximal of the distal-lumen aperture. The proximal lumen has a proximal-lumen aperture in the side of the catheter tube proximal of the medial-lumen aperture. The side aperture of the catheter tube is between the distal-lumen aperture and the medial-lumen aperture, between the medial-lumen aperture and the proximal-lumen aperture, or proximal of the proximal-lumen aperture.

Also disclosed herein is a method for inserting a RICC into a blood-vessel lumen of a patient. The method includes, in some embodiments, a RICC assembly-obtaining step, a needle tract-establishing step, a RICC-advancing step, and an introducer needle-withdrawing step. The RICC assembly-obtaining step includes obtaining a RICC assembly. The RICC assembly includes the RICC and an introducer. The introducer includes a retractable-needle device and a syringe coupled together by a coupling hub. The retractable-needle device includes an introducer needle having a shaft extending through a longitudinal through hole of the coupling hub, through a side aperture in a distal-end portion of a catheter tube of the RICC, along an introducing lumen of the catheter tube, and beyond a distal end of the RICC in at least a ready-to-deploy state of the RICC assembly. The needle tract-establishing step includes establishing a needle tract from an area of skin to the blood-vessel lumen of the patient with a needle tip of the introducer needle while holding a distal-end portion of the retractable-needle device. The RICC-advancing step includes advancing a distal-end portion of the catheter tube into the blood-vessel lumen over the needle tip. The introducer needle-withdrawing step includes withdrawing the shaft of the introducer needle from the introducing lumen by way of the side aperture of the catheter tube.

In some embodiments, the needle tract-establishing step includes ensuring blood flashes back into a sealed blood-flashback chamber of the coupling hub. A cutout of the shaft of the introducer needle is disposed in the blood-flashback chamber for releasing blood into the blood-flashback chamber upon accessing the blood-vessel lumen of the patient.

In some embodiments, the method further includes a blood aspirating-step. The blood aspirating-step includes aspirating blood with the syringe after the needle tract-establishing step but before the introducer needle-withdrawing step. The syringe is fluidly coupled to the blood-flashback chamber by a side-arm lumen of a side arm of the coupling hub for the blood aspirating-step. The blood aspirating-step confirms the needle tip is disposed in the blood-vessel lumen of the patient.

In some embodiments, the RICC-advancing step includes advancing the catheter tube into the blood-vessel lumen with a single finger of a hand while holding a distal-end portion of the retractable-needle device between a thumb and another finger or fingers of the hand. The coupling hub includes a tab configured for advancing the catheter tube into the blood-vessel lumen with the single finger.

In some embodiments, the method further an access guidewire-advancing step. The access guidewire-advancing step includes advancing a distal-end portion of an access guidewire disposed in a needle lumen of the introducer needle into the blood-vessel lumen. The distal-end portion of the access guidewire is advanced by sliding a slider slidably disposed in a longitudinal slot of a housing of the retractable-needle device. The slider includes an extension coupled to a proximal-end portion of the access guidewire disposed in a cavity of the retractable-needle device enclosed by the housing. The access guidewire-advancing step is performed before the RICC-advancing step.

In some embodiments, the introducer needle-withdrawing step includes actuating an introducer-needle actuator of the retractable-needle device. A proximal-end portion of the introducer needle is disposed in a carriage of the retractable-needle device configured to thrust proximally into the cavity of the retractable-needle device when a compressed compression spring around the carriage is released by the introducer-needle actuator.

In some embodiments, the method further a maneuver guidewire-advancing step. The maneuver guidewire-advancing step includes advancing a maneuver guidewire into the blood-vessel lumen by way of a distal lumen having a distal-lumen aperture in the distal end of the RICC. The introducing lumen of the catheter tube is coincident with a distal-end portion of the distal lumen, which mandates performing the introducer needle-withdrawing step before the maneuver guidewire-advancing step.

In some embodiments, the method further includes a sterile barrier-removing step. The sterile barrier-removing step includes removing a sterile barrier disposed over the catheter tube between a catheter hub of the RICC and the side aperture of the catheter tube. The sterile barrier is removed by pulling a sterile-barrier tab of a proximal-end portion of the sterile barrier away from the catheter tube to split the sterile barrier apart.

In some embodiments, the catheter tube includes a first section formed of a first material having a first durometer and a second section proximal of the first section formed of a second material having a second durometer less than the first durometer. The first section of the catheter tube is configured with a column strength for advancing the catheter tube into the blood-vessel lumen.

These and other features of the concepts provided herein will become more apparent to those of skill in the art in view of the accompanying drawings and following description, which describe particular embodiments of such concepts in greater detail.

### DRAWINGS

FIG. 1 illustrates an oblique view of a RICC assembly including a RICC and an introducer in accordance with some embodiments.

FIG. 2 illustrates a top view of the RICC assembly in accordance with some embodiments.

FIG. 3 illustrates a side view of the RICC assembly in accordance with some embodiments.

FIG. 4 illustrates a longitudinal cross section of the RICC assembly in accordance with some embodiments.

FIG. 5 illustrates a distal-end portion of a catheter tube of the RICC in accordance with some embodiments.

FIG. 6 illustrates a first transverse cross section of the catheter tube in accordance with some embodiments.

FIG. 7 illustrates a second transverse cross section of the catheter tube in accordance with some embodiments.

FIG. 8 illustrates a third or fourth transverse cross section of the catheter tube in accordance with some embodiments.

FIG. 9 illustrates a sterile barrier over a maneuver guidewire of the RICC assembly in accordance with some embodiments.

FIG. 10 illustrates a sterile barrier over the catheter tube in accordance with some embodiments.

FIG. 11 illustrates a plunger extension of a syringe of the introducer in accordance with some embodiments.

FIG. 12 illustrates a clip of the introducer in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, there is a need to reduce the number of steps and medical devices involved in introducing a catheter such as a CVC into a patient and advancing the catheter through a vasculature thereof.

Disclosed herein are RICCs including catheter assemblies and methods thereof that address the foregoing. However, it should be understood the RICCs are but one type of catheter in which the concepts provided herein can be embodied or otherwise incorporated. Indeed, peripherally inserted central catheters (“PICCs”), dialysis catheters, or the like can also embody or otherwise incorporate the concepts provided herein for the RICCs, as well as catheter assemblies and methods thereof.

### RICC Assemblies

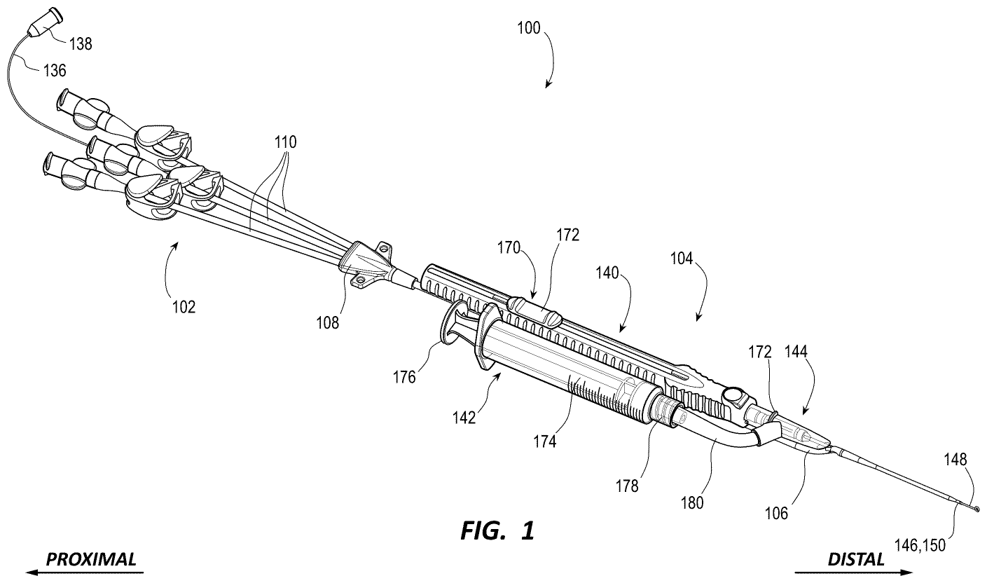

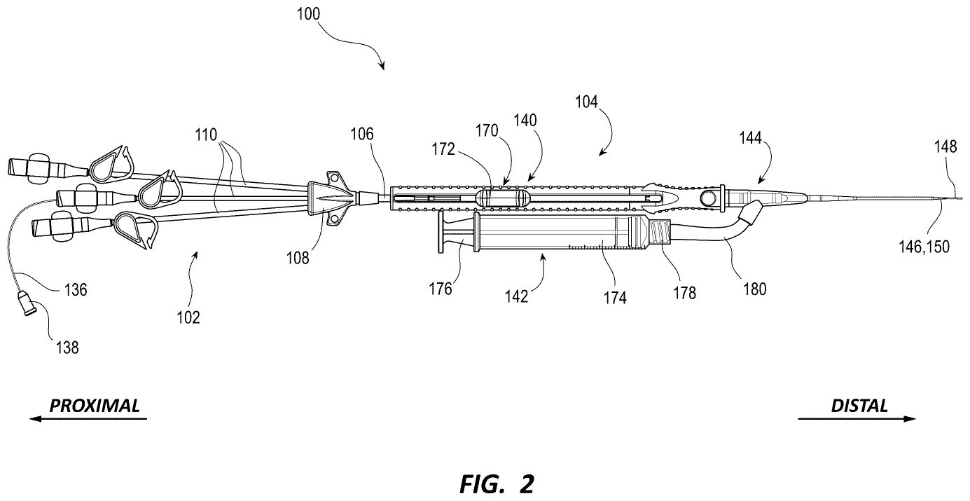

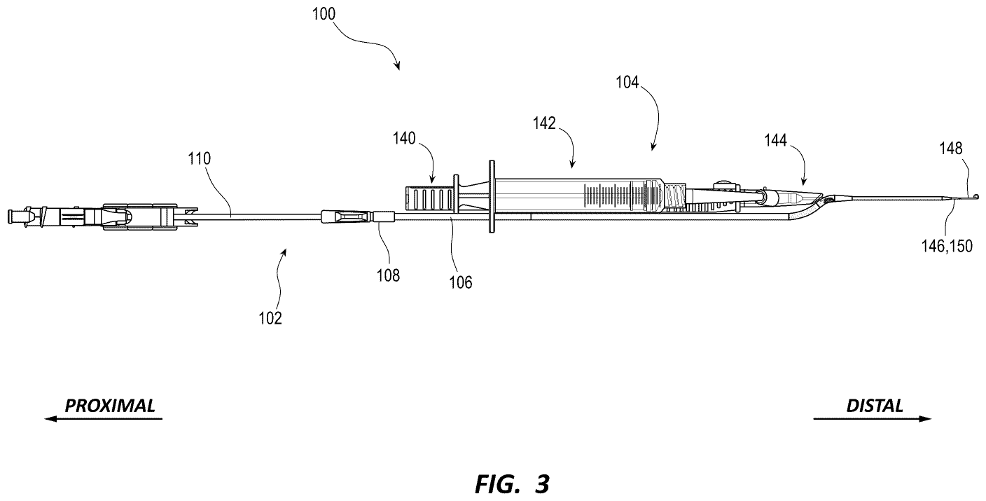

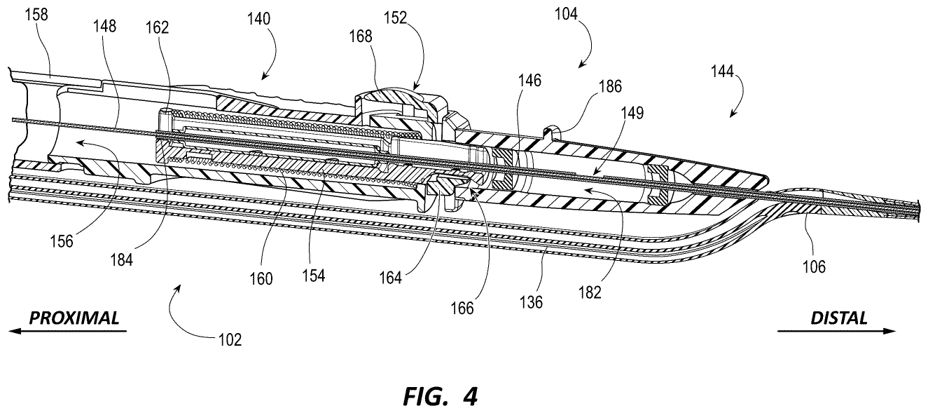

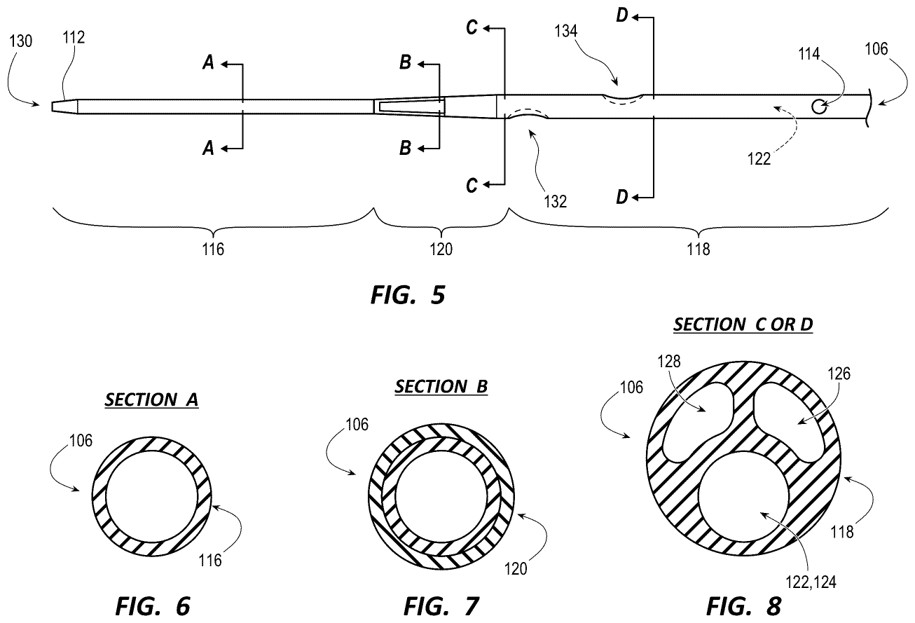

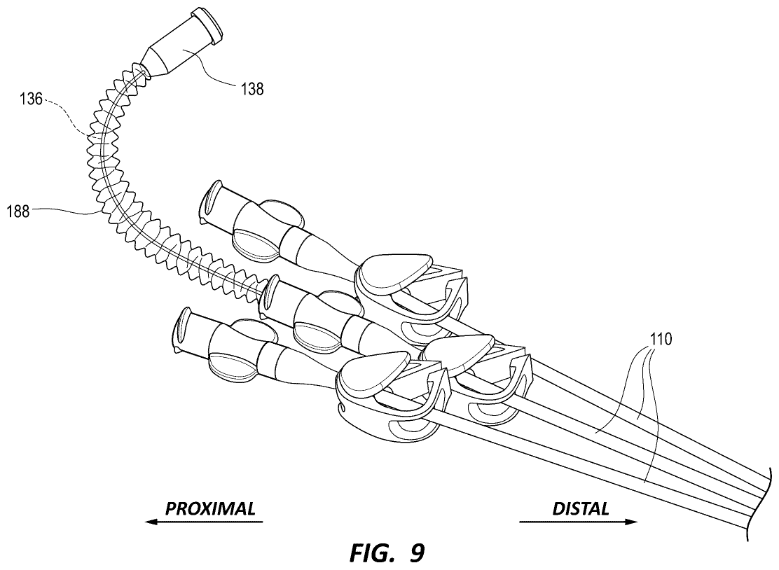

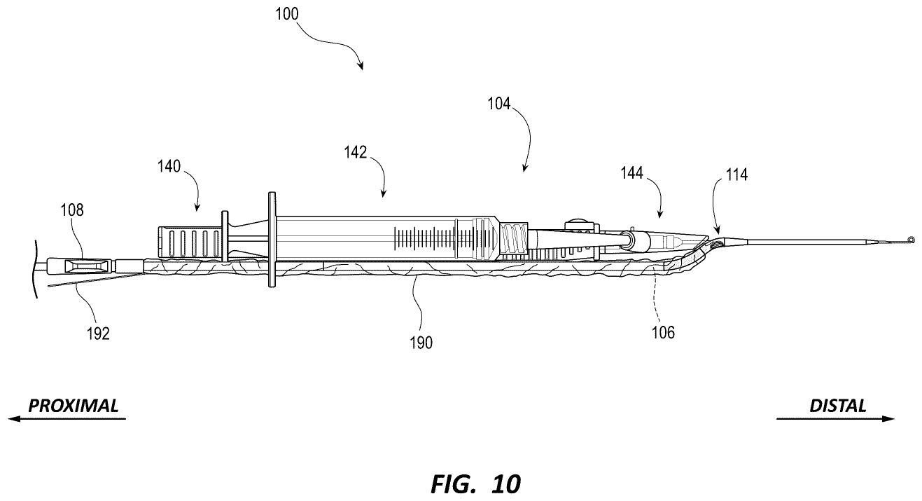

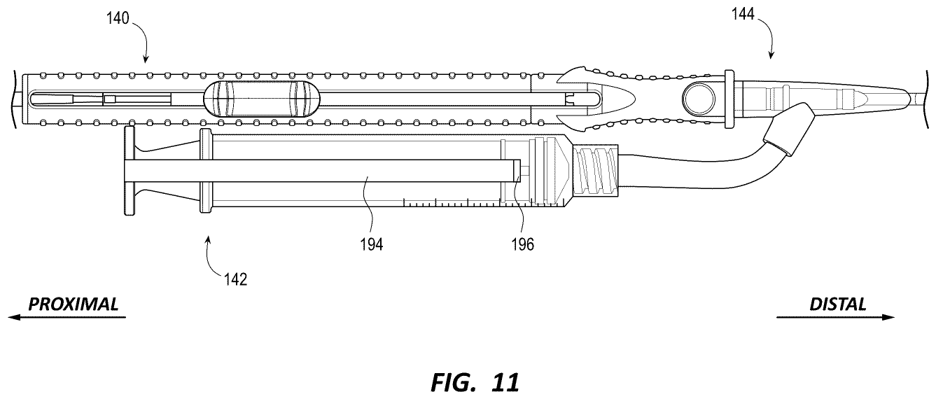

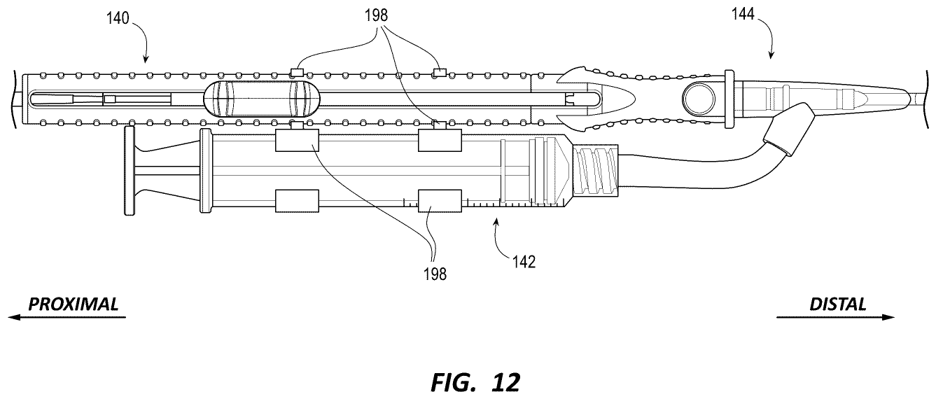

FIGS. 1-3 illustrate various views of a RICC assembly 100 including a RICC 102 and an introducer 104 in accordance with some embodiments. FIG. 4 illustrates a longitudinal cross section of the RICC assembly 100 in accordance with some embodiments. FIG. 5 illustrates a distal-end portion of a catheter tube 106 of the RICC 102 in accordance with some embodiments. FIGS. 6-8 illustrate various transverse cross-sections of the catheter tube 106 in accordance with some embodiments. FIGS. 9-12 illustrate various other features of the RICC assembly 100 in accordance with some embodiments.

As shown, the RICC assembly 100 includes, in some embodiments, the RICC 102 and the introducer 104. The RICC 102 and the introducer 104 are described, in turn, in sections set forth below; however, some crossover between the sections for the RICC 102 and the introducer 104 exist in view of the interrelatedness of the RICC 102 and the introducer 104 in the RICC assembly 100.

The RICC 102 includes the catheter tube 106, a catheter hub 108, and one or more extension legs 110.

The catheter tube 106 includes two or more sections including a catheter tip 112 in a distal-end portion of the catheter tube 106, one or more catheter-tube lumens, and a side aperture 114 through a side of the catheter tube 106 in the distal-end portion of the catheter tube 106.

The two or more sections of the catheter tube 106 can be a main body of the catheter tube 106 and the catheter tip 112, which can be formed as a single extruded piece of a single material or a single coextruded piece of two similar materials. Alternatively, the main body of the catheter tube 106 and the catheter tip 112 can be formed as two different extruded pieces of two similar materials and subsequently coupled. However, FIG. 5 illustrates an embodiment of the catheter tube 106 in which the catheter tube 106 is formed as two different extruded pieces of two different materials and subsequently coupled. Indeed, the catheter tube 106 includes a first section 116 including the catheter tip 112, a second section 118 including the side aperture 114, and an optional transition section 120 therebetween depending upon the manner in which the first section 116 and the second section 118 of the catheter tube 106 are coupled. For example, the first and second sections 116 and 118 of the catheter tube 106 can be bonded by heat, solvent, or adhesive such that the first and second sections 116 and 118 abut each other, or the second section 118 can be inserted into the first section 116 and bonded thereto by heat, solvent, or adhesive, thereby forming the transition section 120. Advantageously, the latter coupling of inserting the second section 118 into the first section 116 facilitates incorporation of a smooth taper into the transition section 120, which taper is useful for dilation during methods of using the RICC assembly 100.

The first section 116 of the catheter tube 106 can be formed of a first material (e.g., a polymeric material such as polytetrafluoroethylene, polypropylene, or polyurethane) having a first durometer, while the second section 118 of the catheter tube 106 can be formed of a second material (e.g., a polymeric material such as polyvinyl chloride, polyethylene, polyurethane, or silicone) having a second durometer less than the first durometer. For example, each section of the first section 116 and the second section 118 of the catheter tube 106 can be made from a different polyurethane having a different durometer. Indeed, polyurethane is advantageous in that polyurethane sections of the catheter tube 106 can be relatively rigid at room-temperature but become more flexible in vivo at body temperature, which reduces irritation to vessel walls and phlebitis. Polyurethane is also advantageous in that can be less thrombogenic than some other polymers.

The catheter tube 106 having at least the first section 116 of the first polymeric material and the second section 118 of the second polymeric material has a column strength sufficient to prevent buckling of the catheter tube 106 when the catheter tube 106 is inserted into an insertion site and advanced through a vasculature of a patient. The column strength of the catheter tube 106 is notable in that it makes it possible to rapidly insert the catheter tube 106 into the insertion site and advance the catheter tube 106 through the vasculature of the patient without using the Seldinger technique.

It should be understood the first durometer and the second durometer can be on different scales (e.g., Type A or Type D), so the second durometer of the second polymeric material might not be numerically less than the first durometer of the first polymeric material. That said, the hardness of the second polymeric material can still be less than the hardness of the first polymeric material as the different scales—each of which ranges from 0 to 100—are designed for characterizing different materials in groups of the materials having a like hardness.

Notwithstanding the foregoing, the first section 116 and the second section 118 of the catheter tube 106 can be formed of a same polymeric material or different polymeric materials having substantially equal durometers provided a column strength of the catheter tube 106 is sufficient to prevent buckling of the catheter tube 106 when inserted into a needle tract to a blood-vessel lumen and advanced through a vasculature of a patient.

The one-or-more catheter-tube lumens can extend through an entirety of the catheter tube 106; however, only one catheter-tube lumen typically extends from a proximal end of the catheter tube 106 to a distal end of the catheter tube 106 in a multiluminal RICC (e.g., a diluminal RICC, a triluminal RICC, a tetraluminal RICC, a pentaluminal RICC, a hexaluminal RICC, etc.). Indeed, the catheter tip 112 typically includes a single lumen therethrough. Optionally, the single lumen through the catheter tip 112 can be referred to as a “tip lumen,” particularly in reference to the first section 116 of the catheter tube 106, which is formed separately from a remainder of the catheter tube 106 and coupled thereto.

Again, the side aperture 114 is through a side of the catheter tube 106 in the distal-end portion of the catheter tube 106; however, the side aperture 114 is proximal of the first section 116 of the catheter tube 106. The side aperture 114 opens into an introducing lumen 122 of the one-or-more catheter-tube lumens. The introducing lumen 122 extends from at least the side aperture 114 in the second section 118 of the catheter tube 106, through the first section 116 of the catheter tube 106 distal thereof, and to a distal end of the RICC 102 (e.g., the distal end of the catheter tube 106 or a distal end of the catheter tip 112). The introducing lumen 122 is coincident with a distal-end portion of the one catheter-tube lumen set forth above that typically extends from the proximal end of the catheter tube 106 to the distal end of the catheter tube 106, particularly the distal-end portion of the foregoing catheter-tube lumen distal of the side aperture 114.

The catheter hub 108 is coupled to a proximal-end portion of the catheter tube 106. The catheter hub 108 includes one or more catheter-hub lumens corresponding in number to the one-or-more catheter-tube lumens. The one-or-more catheter-hub lumens extend through an entirety of the catheter hub 108 from a proximal end of the catheter hub 108 to a distal end of the catheter hub 108.

Each extension leg of the one-or-more extension legs 110 is coupled to the catheter hub 108 by a distal-end portion thereof. The one-or-more extension legs 110 respectively include one or more extension-leg lumens, which, in turn, correspond in number to the one-or-more catheter-tube lumens. Each extension-leg lumen of the one-or-more extension-leg lumens extends through an entirety of the extension leg from a proximal end of the extension leg to a distal end of the extension leg.

Each extension leg of the one-or-more extension legs 110 typically includes a Luer connector coupled to the extension leg, through which Luer connector the extension leg and the extension-leg lumen thereof can be connected to another medical device.

While the RICC 102 can be a monoluminal or multiluminal RICC (e.g., a diluminal RICC, a triluminal RICC, a tetraluminal RICC, a pentaluminal RICC, a hexaluminal RICC, etc.), the RICC 102 shown in FIGS. 1-8 is triluminal including a set of three lumens. The set of three lumens includes, for example, a distal lumen 124, a medial lumen 126, and a proximal lumen 128 formed of fluidly connected portions of three catheter-tube lumens, three hub lumens, and three extension-leg lumens. Whether the RICC 102 is monoluminal or multiluminal, the RICC 102 includes at least the distal lumen 124. The distal lumen 124 includes at least the one catheter-tube lumen set forth above that typically extends from the proximal end of the catheter tube 106 to the distal end of the catheter tube 106 as a catheter tube-lumen portion of the distal lumen 124, as well as a fluidly connected hub- and extension leg-lumen portions of the distal lumen 124. In accordance with the foregoing catheter-tube lumen, the introducing lumen 122 of the catheter tube 106 is coincident with a distal-end portion of the distal lumen 124, particularly the distal-end portion of the distal lumen 124 distal of the side aperture 114.

Further with respect to the triluminal embodiment of the RICC 102, the distal lumen 124 has a distal-lumen aperture 130 in the distal end of the RICC 102 (e.g., the distal end of the catheter tube 106 or the distal end of the catheter tip 112). The medial lumen 126 has a medial-lumen aperture 132 in the side of the catheter tube 106 proximal of the distal-lumen aperture 130 and distal of the following proximal-lumen aperture such that the medial lumen 126 is between the distal-lumen aperture 130 and the proximal-lumen aperture. The proximal lumen 128 has a proximal-lumen aperture 134 in the side of the catheter tube 106 proximal of the medial-lumen aperture 132. The side aperture 114 of the catheter tube 106 can be between the distal-lumen aperture 130 and the medial-lumen aperture 132, between the medial-lumen aperture 132 and the proximal-lumen aperture 134, or proximal of the proximal-lumen aperture 134 as shown in FIG. 5 such that each lumen aperture of the distal-lumen aperture 130, the medial-lumen aperture 132, and the proximal-lumen aperture 134 is distal of the side aperture 114.

The RICC 102 can further include a maneuver guidewire 136. While not shown, the maneuver guidewire 136 can include an atraumatic tip (e.g., a coiled or partially coiled tip) and a length sufficient for advancing the maneuver guidewire 136 to the lower ¼ of the superior vena cava (“SVC”) of the heart. The maneuver guidewire 136 can be captively disposed in the RICC 102 in at least a ready-to-deploy state of the RICC assembly 100. For example, the maneuver guidewire 136 can be disposed in the distal lumen 124 of the RICC 102 with a proximal-end portion or a medial portion of the maneuver guidewire 136 disposed in the extension leg-lumen portion of the distal lumen 124, the medial portion or a distal-end portion of the maneuver guidewire 136 disposed in the hub-lumen portion of the distal lumen 124, and the distal-end portion of the maneuver guidewire 136 disposed in the catheter tube-lumen portion of the distal lumen 124, which is formed of the one catheter-tube lumen set forth above that typically extends from the proximal end of the catheter tube 106 to the distal end of the catheter tube 106. However, the distal-end portion of the foregoing catheter-tube lumen distal of the side aperture 114 is coincident with the introducing lumen 122, which, as set forth below, is occupied by the introducer needle 146 in at least the ready-to-deploy state of the RICC assembly 100. Due to the presence of the introducer needle 146 in the introducing lumen 122, a distal end of the maneuver guidewire 136 is just short of the side aperture 114 in at least the ready-to-deploy state of the RICC assembly 100.

The maneuver guidewire 136 can include a stop 138 (e.g., a hub, a ball, a slug, etc.) about a proximal-end portion of the maneuver guidewire 136 forming a stop end (e.g., a hub end, a ball end, a slug end, etc.) of the maneuver guidewire 136. The stop end of the maneuver guidewire 136 is larger than a proximal-end opening of the distal lumen 124 or the extension leg-lumen portion thereof, thereby providing a distal limit for advancing the maneuver guidewire 136 into the RICC 102. In addition, the maneuver guidewire 136 can be disposed in a fixed-length sterile barrier 188 (e.g., a longitudinal bag) including a closed or sealed proximal end and an otherwise open distal end removably coupled (e.g., removably adhered) to a proximal end of the Luer connector of the extension leg for manual removal of both the sterile barrier and the maneuver guidewire 136 when needed. A combination of the fixed length of the sterile barrier, the closed or sealed proximal end of the sterile barrier, and the distal end of the sterile barrier coupled to the Luer connector provides a limited tract within which the maneuver guidewire 136 can proximally move, thereby providing a proximal limit for withdrawing the maneuver guidewire 136 from the RICC 102. The proximal limit keeps the atraumatic tip of the maneuver guidewire 136 in the distal lumen 124 where, in at least the embodiment of the atraumatic tip having the coiled or partially coiled tip, the atraumatic tip remains in a straightened or uncoiled state, as discussed, for example, in U.S. Pat. Pub. No. 2015/0038943 and U.S. Pat. Pub. No. 2016/0015943, each of which is incorporated by reference in its entirety into this application. This is advantageous for it can be particularly difficult to reinsert such a guidewire in a lumen of a medical device such as a catheter.

Optionally, the stop end of the maneuver guidewire 136 is coupled (e.g., adhered) to the proximal end of the sterile barrier to maintain the stop end of the maneuver guidewire 136 in the proximal end of the sterile barrier, thereby reducing a mismatch between a length of the proximal-end portion of the maneuver guidewire 136 extending beyond the proximal end of the RICC 102 (e.g., a proximal end of the Luer connector) and an unpleated length of the sterile barrier. Reducing the mismatch between the foregoing lengths reduces a likelihood of losing the stop end of the maneuver guidewire 136 in a medial portion of the sterile barrier, which could require time and effort to rematch that would be better spent focusing on the patient.

In addition to providing the proximal limit for withdrawing the maneuver guidewire from the RICC 102, the sterile barrier is configured to maintain sterility of the maneuver guidewire 136 both before use (e.g., shipping and handling, storage, etc.) of the RICC assembly 100 and during use of the RICC assembly 100. During use of the RICC assembly 100, the sterile barrier is configured to provide a no-touch advancing means for advancing the maneuver guidewire 136 into a blood-vessel lumen of a patient upon establishing a needle tract thereto. Likewise, the sterile barrier is configured to provide a no-touch withdrawing means for withdrawing the maneuver guidewire 136 from the blood-vessel lumen of the patient, for example, after the catheter tube 106 has been advanced over the maneuver guidewire 136.

While not shown, the RICC 102 can further include stiffening stylets such as a stylet in either lumen or both lumens of the medial lumen 126 and the proximal lumen 128 of the triluminal embodiment of the RICC 102 for stiffening the RICC 102, thereby providing additional column strength to prevent buckling of the catheter tube 106 when the catheter tube 106 is inserted into an insertion site and advanced through a vasculature of a patient.

The RICC 102 can further include a sterile barrier 190 (e.g., a bag, a casing, etc.) configured to maintain sterility of the catheter tube 106 between the catheter hub 108 and the side aperture 114 of the catheter tube 106 prior to insertion of the catheter tube 106 into a blood-vessel lumen of a patient. When present, the sterile barrier 190 is over the catheter tube 106 between the catheter hub 108 and the side aperture 114 of the catheter tube 106 in at least the ready-to-deploy state of the RICC assembly 100. The sterile barrier 190 is configured to split apart when a sterile-barrier tab 192 of a proximal-end portion of the sterile barrier 190 is pulled away from the catheter tube 106, thereby providing a no-touch mechanism for removing the sterile barrier 190 from the catheter tube 106.

The introducer 104 includes a safety-needle device such as a retractable-needle device 140, a syringe 142, and a coupling hub 144 configured to couple the retractable-needle device 140 and the syringe 142 together. Excepting, for example, a distal-end portion of the introducer needle 146 set forth below, the introducer 104 is proximal of the side aperture 114 of the catheter tube 106 in at least the ready-to-deploy state of the RICC assembly 100 as shown in FIG. 4.

The safety-needle device is configured for ensuring safety of a clinician from injury (e.g., inadvertent needle sticks), contamination, or both while using the RICC assembly 100. For example, the retractable-needle device 140 includes an introducer needle 146, an introducer-needle actuating mechanism configured to retract the introducer needle 146, an access guidewire 148, and an access-guidewire actuating mechanism configured to advance or withdraw the access guidewire 148. One example of an introducer-needle actuating mechanism that retracts the introducer needle 146 to prevent injury, and an access-guidewire actuating mechanism withdraws the access guidewire 148 to prevent contamination is the AccuCath Ace™ Intravascular Catheter System (Becton, Dickinson and Company (BD), Franklin Lakes, New Jersey), aspects of which are disclosed in U.S. Pat. Nos. 8,728,035, 9,162,037, and 10,220,191, each of which is incorporated by reference in its entirety into this application. The safety-needle device can alternatively be based upon design principles of the PowerGlide® Midline Catheter products such as the PowerGlide PRO™ Midline Catheter (BD, Franklin Lakes, New Jersey), aspects of which are disclosed in U.S. Pat. Nos. 8,721,546, 8,932,258, 8,986,227, 8,998,852, 9,757,540, 9,872,971, 9,950,139, 10,086,171, 10,384,039, and 10,426,931, each of which is incorporated by reference in its entirety into this application.

The introducer needle 146 has a shaft, a needle lumen along a length of the shaft, a cutout 149 in a side of the shaft that opens into the needle lumen, and a needle tip 150 (e.g., a beveled tip) in a distal-end portion of the shaft. In at least the ready-to-deploy state of the RICC assembly 100, a proximal-end portion of the shaft including the cutout 149 is disposed in the blood-flashback chamber 182 of the coupling hub 144 set forth below, whereas the distal-end portion of the shaft extends through the longitudinal through hole of the coupling hub 144 set forth below, through the side aperture 114 of the catheter tube 106, and along the introducing lumen 122 of the catheter tube 106 in the ready-to-deploy state of the RICC assembly 100. The needle tip 150 extends beyond the distal end of the RICC 102 when the RICC assembly 100 is in at least the ready-to-deploy state of the RICC assembly 100.

The introducer-needle actuating mechanism includes an introducer-needle actuator 152 and a carriage 154 disposed in a cavity 156 enclosed or otherwise defined by a body-forming housing 158 of the retractable-needle device 140. A proximal-end portion of the introducer needle 146 (e.g., the proximal-end portion of the shaft) is disposed in the carriage 154 as well. The carriage 154 can be spring loaded with a compressed compression spring 160 around the carriage 154, wherein the compression spring 160 is compressed between a proximal flange 162 of the carriage 154 and a distal-end piece of the retractable-needle device 140 (e.g., a distal wall of the housing 158) in at least the ready-to-deploy state of the RICC assembly 100. The introducer-needle actuator 152 can include a catch 164 configured to hold the spring-loaded carriage 154 by a complementary recess 166 in a coupling hub-connecting portion of the carriage 154 extending through a distal end of the retractable-needle device 140 or the housing 158 thereof. When the foregoing introducer-needle actuator 152 is toggled, for example, by a button 168 of the introducer-needle actuator 152, the introducer-needle actuator 152 dislodges the catch 164 from the recess 166 in the coupling hub-connecting portion of the carriage 154, thereby releasing the spring-loaded carriage 154 and the compression spring 160 therearound. Releasing the spring-loaded carriage 154 allows the compressed compression spring 160 to relax, which allows potential energy stored by the compressed compression spring 160 to be released. When relaxing, the compression spring 160 proximally extends and thrusts both the carriage 154 and the introducer needle 146 into the cavity 156 of the retractable-needle device 140.

The access guidewire 148 is captively disposed in a combination of the cavity 156 of the retractable-needle device 140 and the needle lumen of the introducer needle 146. In at least the ready-to-deploy state of the RICC assembly 100, a proximal-end portion of the access guidewire 148 is disposed in the cavity 156 and a distal-end portion of the access guidewire 148 is disposed in the needle lumen. Advantageously, a majority of the access guidewire 148 is contained within the retractable-needle device 140 during most if not all operating states of a number of operating states of the RICC assembly 100. Containing the access guidewire 148 within the retractable-needle device 140 also contains any blood or other bodily fluids within the retractable-needle device 140 that result from withdrawing the access guidewire 148 from a blood-vessel lumen of a patient. This minimizes or prevents a potential for contaminating an operating field or any clinicians operating within the operating field with the blood or other bodily fluids.

The access-guidewire actuating mechanism includes an access-guidewire actuator 170 coupled to the proximal-end portion of the access guidewire 148 disposed within the housing 158 of the retractable-needle device 140. For example, the access-guidewire actuator 170 includes a slider 172 slidably disposed in a closed-ended longitudinal slot of the housing 158 of the retractable-needle device 140, the closed ends of the longitudinal slot providing distal and proximal stops for advancing and withdrawing the access guidewire 148, respectively. While not shown, the slider 172 can include an extension extending into the cavity 156 of the retractable-needle device 140 where it is coupled to the proximal-end portion of the access guidewire 148. The foregoing access-guidewire actuator 170 is configured to advance the distal-end portion of the access guidewire 148 beyond the needle tip 150 when the access-guidewire actuator 170 is distally moved in accordance with the longitudinal slot. Likewise, the access-guidewire actuator 170 is configured to withdraw the access guidewire 148 into the retractable-needle device 140 such as the distal-end portion of the access guidewire 148 into the distal-end portion of the shaft or needle lumen proximal of the needle tip 150 when the access-guidewire actuator 170 is proximally moved in accordance with the longitudinal slot.

The access-guidewire actuator 170 can be actuated to withdraw the access guidewire 148 into the retractable-needle device 140 by manual actuation as set forth above or automatic actuation. With respect to automatic actuation, the carriage 154 of the introducer-needle actuating mechanism can be configured to engage the access-guidewire actuator 170 when the carriage 154 is thrust proximally into the cavity 156 of the retractable-needle device 140 by the compression spring 160 as the compression spring 160 relaxes. For example, a proximal end of the carriage 154 can be configured to engage a distal end of the slider 172 or the extension thereof when the carriage 154 is thrust proximally into the cavity 156 of the retractable-needle device 140 by the compression spring 160. The access guidewire 148 is withdrawn into the cavity 156 of the retractable-needle device 140 by the distal-end portion thereof together with the introducer needle 146 when the introducer-needle actuator 152 is automatically actuated.

The syringe 142 includes a barrel 174, a plunger 176 disposed in the barrel 174, and a syringe tip 178 extending from a distal end of the barrel 174, which is fluidly coupled to the side arm 180 of the coupling hub 144 set forth below at least when the RICC assembly 100 is in at least the ready-to-deploy thereof.

As shown, the plunger 176 can include a plunger extension 194 distally extending from a distal-end portion of the plunger along the barrel 174 of the syringe. The plunger extension 194 includes a tab 196 configured to allow a clinician to withdraw the plunger 176 from the barrel 174 of the syringe 142 by proximally pushing the tab 196 such as during the blood-aspirating step set forth below. Such a configuration for withdrawing the plunger 176 from the barrel 174 is advantageous when handling the RICC assembly 100 around the coupling hub 144, which is a preferred position for handling the RICC assembly 100 to minimize large movements in a distal-end portion of the RICC 102. Indeed, the more proximal the position in which the RICC assembly 100 is handled, the larger otherwise small, inadvertent movements of the hand will be in the distal-end portion of the RICC 102.

As set forth above, the coupling hub 144 is configured to couple the retractable-needle device 140 and the syringe 142 together such as in the ready-to-deploy state of the RICC assembly 100. The coupling hub 144 includes a carriage connector configured to connect to the coupling hub-connecting portion of the carriage 154, a side arm 180 extending from a side of the coupling hub 144, and a side-arm connector of the side arm 180 configured to connect to the syringe tip 178 of the syringe 142.

The coupling hub 144 is translucent and preferably colorless for observing blood flashback from a venipuncture with the introducer needle 146. Indeed, the coupling hub 144 includes a sealed blood-flashback chamber 182 proximal of a longitudinal through hole of the coupling hub 144 for observing blood flashback. As set forth above, the proximal-end portion of the shaft of the introducer needle 146 including the cutout 149 is disposed in the blood-flashback chamber 182, which cutout 149 is configured to release blood into the blood-flashback chamber 182 upon the needle tip 150 of the introducer needle 146 accessing a blood-vessel lumen of a patient. In addition, the side arm 180 of the coupling hub 144 includes a side-arm lumen that fluidly couples the syringe 142 to the blood-flashback chamber 182 in at least the ready-to-deploy state of the RICC assembly 100. Once blood flashback is observed in the blood-flashback chamber 182 from a venipuncture with the introducer needle 146, the syringe 142 can be used to aspirate additional blood for confirmation of access to the blood-vessel lumen. Seals (e.g., gaskets) at proximal- and distal-end portions of the blood-flash chamber, as well as a seal 184 (e.g., a gasket) about a proximal end of the shaft of the introducer needle 146 through which a bare, unwound portion of the access guidewire 148 passes, enable aspiration of the additional blood with the syringe 142.

The coupling hub 144 can also include a tab 186 in a distal-end portion of the coupling hub 144. The tab 186 is configured to allow a clinician to single handedly advance the RICC 102 over the needle tip 150 with a single finger of a hand while holding a distal-end portion of the retractable-needle device 140 between a thumb and another finger or fingers of the same hand. The tab 186 facilitates a no-touch mechanism for advancing the RICC 102, specifically the distal-end portion of the catheter tube 106, over the needle tip 150, the distal-end portion of the access guidewire 148, or a combination thereof and into a blood-vessel lumen of a patient.

The coupling hub 144 including the side arm 180 thereof can be rigid for holding the syringe 142 in place alongside the retractable-needle device 140 in the RICC assembly 100 in at least the ready-to-deploy state of the RICC assembly 100. That said, the side arm 180 of the foregoing can alternatively be flexible in some embodiments. In such embodiments, the RICC assembly 100 or the introducer 104 thereof includes a clip 198 having a syringe-clipping portion configured to clip the syringe 142 by the barrel 174 thereof and a needle device-clipping portion configured to clip the retractable-needle device 140 by the body-forming housing 158 thereof. The needle device-clipping portion of the clip 198 is configured to allow the retractable-needle device 140 to slide therein. In other words, the retractable-needle device 140 is slidably disposed in the needle device-clipping portion of the clip 198, which allows for the introducer needle-withdrawing step set forth below.

FIGS. 1-4 illustrate the RICC assembly 100 in at least the ready-to-deploy state thereof. When the RICC assembly 100 is in the ready-to-deploy state, little more than the needle tip 150 of the introducer needle 146 extends from the distal end of the RICC 102 for a venipuncture. Indeed, a distal-end portion (e.g., about 7 cm) of the shaft of the introducer needle 146 extends through the longitudinal through hole of the coupling hub 144, through the side aperture 114 of the catheter tube 106, along the introducing lumen 122 of the catheter tube 106, and through the distal end of the RICC 102 when the RICC assembly 100 is in at least the ready-to-deploy state thereof. However, in some embodiments, 1-3 cm or more of the distal-end portion of the shaft can extend from the distal end of the RICC 102 the venipuncture. In such embodiments, the first section 116 of the catheter tube 106 is shorter in length as opposed to the shaft being longer in length. Additional operating states of the number of operating states of the RICC assembly 100 can be discerned from functional aspect of the RICC assembly 100 set forth above or steps of the method for inserting the RICC 102 set forth below.

### Methods

A method of the RICC assembly 100 includes a method for inserting the RICC 102 into a blood-vessel lumen of a patient. Such a method includes, in some embodiments, a RICC assembly-obtaining step, a needle tract-establishing step, a RICC-advancing step, and an introducer needle-withdrawing step.

The RICC assembly-obtaining step includes obtaining the RICC assembly 100. As set forth above, the RICC assembly 100 includes the RICC 102 and the introducer 104. The introducer 104 includes the retractable-needle device 140 and the syringe 142 coupled together by the coupling hub 144.

The method can further include a needle tip-ensuring step of ensuring the needle tip 150 of the introducer needle 146 extends from the distal end of the RICC 102 before the needle tract-establishing step. As set forth above, the retractable-needle device 140 includes the introducer needle 146 having the shaft extending through the longitudinal through hole of the coupling hub 144, through the side aperture 114 in the distal-end portion of the catheter tube 106 of the RICC 102, along the introducing lumen 122 of the catheter tube 106, and beyond the distal end of the RICC 102 in at least the ready-to-deploy state of the RICC assembly 100.

The needle tract-establishing step includes establishing a needle tract from an area of skin to the blood-vessel lumen of the patient with the needle tip 150 of the introducer needle 146 while holding the distal-end portion of the retractable-needle device 140. The needle tract-establishing step can also include ensuring blood flashes back into the sealed blood-flashback chamber 182 of the coupling hub 144. As set forth above, the cutout 149 of the shaft of the introducer needle 146 is disposed in the blood-flashback chamber 182 for releasing blood into the blood-flashback chamber 182 upon accessing the blood-vessel lumen of the patient.

The method can further include a blood aspirating-step. The blood aspirating-step includes aspirating blood with the syringe 142 after the needle tract-establishing step but before the introducer needle-withdrawing step. Again, the syringe 142 is fluidly coupled to the blood-flashback chamber 182 of the coupling hub 144 by the side-arm lumen of the side arm 180 for the blood aspirating-step. The blood aspirating-step confirms the needle tip 150 is disposed in the blood-vessel lumen of the patient.

The method can further include an access guidewire-advancing step. The access guidewire-advancing step includes advancing the distal-end portion of the access guidewire 148 from the needle lumen of the introducer needle 146 into the blood-vessel lumen, which facilitates first-stick success by making the access guidewire 148 immediately available before the blood-lumen vessel can be lost due to small inadvertent movements of the RICC assembly 100. The distal-end portion of the access guidewire 148 is advanced, in some embodiments, by distally sliding the slider 172 in the longitudinal slot of the housing 158 of the retractable-needle device 140. As set forth above, the slider 172 can include an extension coupled to the proximal-end portion of the access guidewire 148 disposed in the cavity 156 defined by the housing 158 of the retractable-needle device 140. The access guidewire-advancing step is performed before the first RICC-advancing step such that the distal-end portion of the catheter tube 106 can be advanced over the access guidewire 148 as well as the needle tip 150 of the introducer needle 146.

The first RICC-advancing step includes advancing the distal-end portion of the catheter tube 106 into the blood-vessel lumen over the needle tip 150, the access guidewire 148, or both. As set forth above, the catheter tube 106 includes the first section 116 formed of the first material having the first durometer and the second section 118 formed of the second material having the second durometer less than the first durometer. The first section 116 of the catheter tube 106 is configured with a column strength for advancing the catheter tube 106 into the blood-vessel lumen over the access guidewire 148 or the maneuver guidewire 136 set forth below provided in the maneuver guidewire-advancing step. For example, the first RICC-advancing step can include advancing the catheter tube 106 into the blood-vessel lumen with a single finger of a hand (e.g., with a flick-type motion of the finger) while holding the distal-end portion of the retractable-needle device 140 between a thumb and another finger or fingers of the same hand. The coupling hub 144 includes the tab 186 configured for advancing the catheter tube 106 into the blood-vessel lumen with the single finger. When the coupling is advanced by the single finger, the coupling hub 144, in turn, advances the catheter tube 106 into the blood-vessel lumen.

The introducer needle-withdrawing step includes withdrawing the shaft of the introducer needle 146 from the blood-vessel lumen of the patient, as well as the introducing lumen 122 of the RICC 102 by way of the side aperture 114 of the catheter tube 106. The introducer needle-withdrawing step includes actuating the introducer-needle actuator 152 (e.g., by the button 168) of the retractable-needle device 140. As set forth above, the proximal-end portion of the introducer needle 146 is disposed in the spring-loaded carriage 154 of the retractable-needle device 140, which is configured to thrust proximally into the cavity 156 of the retractable-needle device 140 when the compressed compression spring 160 around the carriage 154 is released by the introducer-needle actuator 152.

The method can further include an access guidewire-withdrawing step of withdrawing the access guidewire 148 from at least the blood-vessel lumen of the patient. The access guidewire-withdrawing step can be performed manually by manual actuation of the access-guidewire actuator 170. For example, the access guidewire 148 can be manually withdrawn from the blood-vessel lumen of the patient by proximally sliding the slider 172 in the longitudinal slot of the housing 158 of the retractable-needle device 140. Alternatively, the access guidewire-withdrawing step can be performed automatically by automatic actuation of the access-guidewire actuator 170. For example, the carriage 154 of the introducer-needle actuating mechanism can be configured to engage the access-guidewire actuator 170 (e.g., the slider 172 or the extension thereof) when the carriage 154 is thrust proximally into the cavity 156 of the retractable-needle device 140 by the compression spring 160. Thus, when the introducer-needle actuator 152 is actuated to release the spring-loaded carriage 154 in the introducer needle-withdrawing step, the access guidewire-withdrawing step is also performed.

Combined, the introducer needle-withdrawing step and the access guidewire-withdrawing step can be considered an introducer-removing step of completely removing the introducer 104 from the RICC 102. Depending upon a length of the access guidewire 148, however, the introducer-removing step can further include separating the introducer 104 from the RICC 102 a distance sufficient to completely withdraw the access guidewire 148 from the introducing lumen 122 of the RICC 102 by way of the side aperture 114 of the catheter tube 106. The introducer-removing step can be performed after the first RICC-advancing step such as after the distal-end portion of the catheter tube 106 is suitably placed within the blood-vessel lumen over both the needle tip 150 and the access guidewire 148.

The method can further a maneuver guidewire-advancing step. The maneuver guidewire-advancing step includes advancing the maneuver guidewire 136 into the blood-vessel lumen by way of, for example, the distal-lumen aperture 130 in the distal end of the RICC 102. As set forth above, the introducing lumen 122 of the catheter tube 106 is coincident with the distal-end portion of the distal lumen 124, particularly the distal-end portion of the distal lumen 124 distal of the side aperture 114. As such, the introducer-removing step of completely removing the introducer 104 from the RICC 102 is mandated before the maneuver guidewire-advancing step to ensure the distal lumen 124, or the introducing lumen 122 thereof, is free of both the introducer needle 146 and the access guidewire 148

The method can further include a second RICC-advancing step of advancing the distal-end portion of the catheter tube 106 farther into the blood-vessel lumen over the maneuver guidewire 136 such as to the SVC. The maneuver guidewire 136 provides the second section 118 of the catheter tube 106 sufficient column strength for the second RICC-advancing step.

The method can further include a sterile barrier-removing step when the sterile barrier is present over the catheter tube 106 between the catheter hub 108 of the RICC 102 and the side aperture 114 of the catheter tube 106. The sterile barrier-removing step includes removing the sterile barrier. The sterile barrier is removed by pulling the sterile-barrier tab of the proximal-end portion of the sterile barrier away from the catheter tube 106 to split the sterile barrier apart.

The method can further include a maneuver guidewire-withdrawing step of withdrawing the maneuver guidewire 136 from the blood-vessel lumen of the patient, as well as completely removing the maneuver guidewire 136 from the RICC 102.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An introducer, comprising:
a retractable-needle device including an introducer needle having a shaft and a needle tip in a distal-end portion of the shaft, a proximal-end portion of the shaft disposed in a carriage of the retractable-needle device; and
a coupling hub, the shaft of the introducer needle extending from the carriage, through a sealed blood-flashback chamber of the coupling hub, and out a longitudinal through hole of the coupling hub distal of the blood-flashback chamber for establishing a needle tract from an area of skin to a blood-vessel lumen of a patient with the needle tip of the introducer needle.

2. The introducer of claim 1, wherein a cutout of the shaft of the introducer needle is disposed in the blood-flashback chamber, the cutout configured to release blood into the blood-flashback chamber upon the needle tip accessing the blood-vessel lumen of the patient.

3. The introducer of claim 2, wherein the blood-flashback chamber is sealed with a seal in a proximal-end portion of the blood-flashback chamber and another seal in a distal-end portion of the blood-flashback chamber.

4. The introducer of claim 1, wherein the coupling hub includes a side arm extending from a side of the coupling hub for coupling the retractable-needle device and another medical device together.

5. The introducer of claim 4, further comprising:
a syringe as the other medical device, the syringe coupled to the side arm of the coupling hub, the syringe fluidly coupled to the blood-flashback chamber by a side-arm lumen of the side arm for aspirating blood upon the needle tip accessing the blood-vessel lumen of the patient.

6. The introducer of claim 5, wherein the syringe includes a plunger disposed in the barrel of the syringe, the plunger including a plunger extension extending from a distal-end portion of the plunger along the barrel of the syringe, thereby allowing a clinician to withdraw the plunger from the barrel by proximally pushing a tab of the plunger extension while handling the introducer around the coupling hub.

7. The introducer of claim 1, the retractable-needle device further including:
an access guidewire disposed in a needle lumen of the introducer needle; and
an access-guidewire actuator configured to advance a distal-end portion of the access guidewire beyond the needle tip or withdraw the distal-end portion of the access guidewire into the distal-end portion of the shaft of the introducer needle proximal of the needle tip.

8. The introducer of claim 7, wherein a bare, unwound portion of the access guidewire passes through a seal about a proximal end of the shaft of the introducer needle, thereby enabling aspiration of blood upon the needle tip accessing the blood-vessel lumen of the patient.

9. The introducer of claim 7, wherein the access-guidewire actuator includes a slider slidably disposed in a longitudinal slot of a housing of the retractable-needle device, the slider including an extension coupled to a proximal-end portion of the access guidewire within a cavity of the retractable-needle device enclosed by the housing.

10. The introducer of claim 9, wherein an introducer-needle actuator of the retractable-needle device is configured to release a compressed compression spring around the carriage when actuated and thrust both the carriage and the introducer needle proximally into the cavity of the retractable-needle device.

11. The introducer of claim 10, wherein the carriage is configured to engage the slider of the access-guidewire actuator when the carriage is thrust proximally into the cavity of the retractable-needle device, thereby withdrawing the access guidewire into the cavity of the retractable-needle device by the distal-end portion thereof together with the introducer needle when the introducer-needle actuator is actuated.

12. The introducer of claim 1, the introducer further comprising:
a clip having a syringe-clipping portion and needle device-clipping portion, the syringe-clipping portion of the clip configured to clip a syringe by a barrel of the syringe and the needle device-clipping portion of the clip configured to clip the retractable-needle device by a body of the retractable-needle device while allowing the retractable-needle device to slide in the needle device-clipping portion of the clip.

13. The introducer of claim 1, wherein the introducer is configured for introducing a rapidly insertable central catheter (“RICC”), the RICC including a catheter tube having a side aperture through a side of the catheter tube in a distal-end portion of the catheter tube, the side aperture opening into an introducing lumen of the catheter tube that extends from at least the side aperture to a distal end of the RICC, and, when the RICC is coupled with the introducer, the shaft of the introducer needle further extends through the introducing lumen of the catheter tube such that the needle tip of the introducer needle extends beyond the distal end of the RICC.

14. A coupling hub of an introducer, comprising:
a sealed blood-flashback chamber including a seal in a proximal-end portion of the blood-flashback chamber and another seal in a distal-end portion of the blood-flashback chamber;
a longitudinal through hole distal of the blood-flashback chamber, the coupling hub configured for extension of a shaft of an introducer needle through both the blood-flashback chamber and the longitudinal through hole with a cutout of the shaft disposed in the blood-flashback chamber; and
a side arm extending from a side of the coupling hub for coupling a retractable-needle device including the introducer needle together with another medical device together.

15. The coupling hub of claim 14, wherein the side arm is rigid.

16. The coupling hub of claim 14, wherein the side arm is flexible.

17. The coupling hub of claim 14, wherein a side-arm lumen of the side arm is fluidly coupled to the blood-flashback chamber for aspirating blood with the other medical device upon a venipuncture with the introducer needle.

18. The coupling hub of claim 14, wherein the coupling hub is translucent for observing blood flashback in the blood-flashback chamber upon a venipuncture with the introducer needle.

19. The coupling hub of claim 18, wherein the coupling hub is colorless.

20. The coupling hub of claim 14, further comprising:
a tab in a distal-end portion of the coupling hub configured to allow a clinician to single handedly advance a rapidly insertable central catheter (“RICC”) over the shaft of the introducer needle with a single finger of a hand while holding a distal-end portion of the retractable-needle device between a thumb and another finger or fingers of the same hand, the tab thereby facilitating a no-touch mechanism for advancing the RICC into a blood-vessel lumen of a patient.
